(19) United States
(12) Patent Application Publication
(10) Pub. No.: US 2018/0064938 A1
(43) Pub. Date: Mar. 8, 2018

(54) ORALLY IMPLANTABLE NEUROSTIMULATOR

(71) Applicant: Regents of the University of Minnesota, Minneapolis, MN (US)

(72) Inventors: Ahmed Mohamed Abdelhalim Selim, Elkhorn, NE (US); Adam Joseph Black, Plymouth, MN (US); Steven David Reinitz, Marlborough, MA (US); Bradley Frank Slaker, Loretto, MN (US)

(21) Appl. No.: 15/698,160

(22) Filed: Sep. 7, 2017

Related U.S. Application Data
(60) Provisional application No. 62/384,496, filed on Sep. 7, 2016.

(51) Int. Cl. A61N 1/36; A61B 5/00

(52) U.S. Cl. CPC A61N 1/3611; A61M 21/02; A61B 5/4818; A61N 1/36078

(57) ABSTRACT

A system for preventing obstruction of an airway includes an internal component and an external component in embodiments described herein. The internal component can include an electrical coupling configured to extend to a muscle or nerve, and an access port electrically connected to the electrical coupling. The external component can include an access port counterpart configured to engage with the access port to provide an electrical connection, and a power supply. The power supply can provide power to the access port counterpart and the electrical connection, thereby powering the electrical coupling at the muscle or nerve to prevent obstruction of an airway due to obstructive sleep apnea.

### RELATED APPLICATION

The present application claims the benefit of U.S. Provisional Application No. 62/384,496 filed Sep. 7, 2016, which is hereby incorporated herein in its entirety by reference.

### TECHNICAL FIELD

Embodiments include neuro-muscular stimulators that can be used to stimulate the hypoglossal nerve and the genioglossus muscle and prevent sleep apnea or breathing obstructions caused by inadequate motor tone of the tongue and/or pharyngeal wall muscles.

### BACKGROUND

Obstructive sleep apnea (OSA) is a sleep disorder that can cause a patient to stop breathing during sleep due to an obstruction in the upper airways. OSA is estimated to affect a quarter of middle-aged men and about 9% of middle-aged women. OSA can be caused by lack of adequate motor tone of the tongue and pharyngeal wall muscles, which leads to collapse of the upper airway. Typically, when this occurs during sleep, a drop in oxygen saturation will lead to short periods in which the person suffering from OSA will wake up.

As a result, the OSA sufferer will experience daytime sleepiness and potentially other health consequences. Among these health consequences are fatigue and hypersomnolence, an increase in incidence of malignant ventricular arrhythmia, higher incidence of stroke, high blood pressure, and other cardiovascular events, adult-onset epilepsy, insulin resistance and development of diabetes, and increased perioperative complications. Therefore, treatment of OSA will not just improve sleep but also has the potential to drastically reduce the likelihood of other serious health conditions.

Currently available treatment for OSA typically includes the use of a continuous positive airway pressure (CPAP) machine. CPAP machines can be uncomfortable to use because they require a mask and high pressured air, and create noise and limit movement. Because of these limitations, patient compliance with a recommended use of CPAP machines is relatively low. Almost a third of patients refuse to use a CPAP machine after being diagnosed with OSA, and only about half of those using a CPAP machine will continue to use it after five years.

Various alternatives, including oral and nasal devices that can be donned prior to sleeping, are available but primarily only used for patients having mild OSA due to more limited effectiveness than CPAP machines. Surgical reconstruction of the airways is also possible in some cases, but this type of surgery is complex and has a limited success rate.

Another alternative to CPAP machines, external devices, and surgical reconstruction, is an implantable neurostimulator. Typically, embedding a neurostimulator requires an extensive, multiple-hours-long surgery, which can involve similar complications to those involved in surgical reconstruction. Furthermore, conventional implantable neurostimulators for treating OSA are expensive and have a high failure rate of up to a third of installed devices.

For the reasons listed above, there is a need for a way to prevent upper airway collapse during sleep in patients with OSA that promotes better patient compliance and is less expensive, less prone to failure, and less likely to result in surgical complications than existing solutions.

### SUMMARY

In embodiments, a neuro-muscular stimulator can be implanted inside the mouth to stimulate the hypoglossal nerve and the genioglossus muscle. The device can have an implantable part, that is the stimulator lead, and an exposed part, which can be positioned in the lower jaw and act as an access point between the implanted stimulator lead and an outside power source and electrical pulse generator. The outside power source and stimulator can be a part of a removable object such as a retainer, which can be donned by the patient at night and removed in the morning.

According to one embodiment, a system for preventing obstruction of an airway includes an internal component having an electrical coupling configured to extend to a muscle or nerve, and an access port electrically connected to the electrical coupling. The system further includes an external component having an access port counterpart configured to engage with the access port to provide an electrical connection, and a power supply. The power supply is configured to provide power to the access port counterpart and the electrical connection, thereby powering the electrical coupling at the muscle or nerve.

In embodiments, the external component can be a retainer. Additionally or alternatively, the power supply can be a battery. Additionally or alternatively, the external component can include a processor. The internal component can include a plurality of electrical couplings each configured to extend to a corresponding muscle or nerve. The processor can be configured to selectively power each of the plurality of electrical couplings. Additionally or alternatively, the access port can be configured to be arranged in a surgically-altered jaw. Additionally or alternatively, the access port can be configured to be arranged in a tooth. Additionally or alternatively, at least one of the electrical couplings can extend to an ending at a hypoglossal nerve. Additionally or alternatively, at least one of the electrical couplings extends to an ending at a genioglossus muscle. In embodiments, the power supply can include a rechargeable battery. In embodiments, the system includes a charging port configured to facilitate charging of the battery. In embodiments, the rechargeable battery can be charged wirelessly. The power supply can be a disposable battery. The external component can include an access panel configured to permit replacement of the disposable battery. The external component can also include a bridge.

According to another embodiment, a kit for treating obstructive sleep apnea includes an internal component having an electrical coupling configured to extend to a muscle or nerve, and an access port electrically connected to the electrical coupling, wherein the internal component is configured to be surgically implanted adjacent at least one nerve or muscle in a patient. The kit also includes an external component having an access port counterpart configured to engage with the access port to provide an electrical connection, and a power supply. In embodiments, the electrical coupling can include a plurality of endings configured to be surgically implanted adjacent to each of a corresponding one of the at least one nerve or muscle of the patient.

According to another embodiment, a retainer for use with a system for treating obstructive sleep apnea includes a body configured to engage with a user's teeth, a bridge coupled to the body, the bridge comprising an electrical connector configured to engage with an access port, a power supply configured to provide power to the electrical connector, the power supply comprising a battery, and a processor configured to direct power from the power supply to the electrical connector at a predetermined level. In embodiments, the charging port is electrically coupled to the power supply such that the power supply can be recharged via the charging port.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a partial cutaway view of a patient's airway including an implantable neurostimulating device according to an embodiment.

FIG. 2 is a perspective view of a jawbone housing an access port to a neurostimulating device according to an embodiment.

FIG. 3 is a bottom view of a retainer for operating a neurostimulating device according to an embodiment.

FIG. 4 is a simplified chart of a system for treating obstructive sleep apnea according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

As shown with respect to the embodiments described herein, a system for treatment of obstructive sleep apnea (OSA) includes both an internal component and an external component. The internal component is an electrically conductive system that can stimulate the hypoglossal nerve and/or the genioglossus muscle to keep the airway of a person suffering from OSA clear. The external component can be coupled to the internal component to provide electrical power for the internal component, as well as provide a driving signal that can be timed to coincide with breathing patterns, prevent muscle fatigue, or otherwise monitor and oversee neurostimulation.

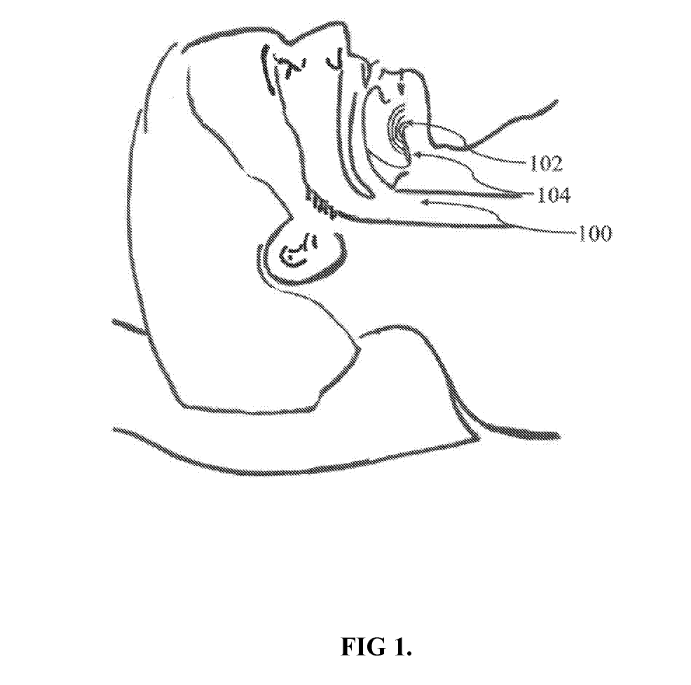

FIG. 1 includes a cross-sectional view of an airway of a person suffering from OSA. The cross-sectional view includes a region 100 of the throat that can become constricted due to insufficient muscle tone of the genioglossus muscle during sleep. FIG. 1 also depicts internal component 102 which is implanted into the person, coupled to electrical coupler 104.

To prevent constriction of region 100 during sleep, internal component 102 can be activated to provide a desired neurostimulating current to the genioglossus muscle or the hypoglossal nerve that leads to it. Therefore, internal component 102 is positioned such that it can be used to stimulate the hypoglossal nerve and/or the genioglossus muscle based on a driving signal.

In embodiments, internal component 102 can be surgically implanted into the patient. By surgically implanting this component into the patient, there is no chance that the patient will stop using the device, as is the case with conventional OSA solutions like CPAP machines and wearable nose or throat products. Internal component 102 can be quite small and have limited or no negative effects on the patient's day-to-day lifestyle.

Internal component 102 can be coupled to a portion of the hypoglossal nerve and/or the genioglossus muscle, or can be coupled to more than one portion. In some embodiments, internal component 102 can be a plate or a series of wires, coupled to a variety of muscles and/or nerves, based on the needs of a particular patient. A physician or surgeon can determine which muscles have insufficient tone or are causing obstruction at region 100, and adjust the placement of internal component 102 so that it stimulates the components that have insufficient tone. Because internal component 102 is surgically implanted, it can be desirable to couple internal component to multiple nerves or muscles if a physician or surgeon believes that any of them may be used in future to prevent OSA as described in more detail below.

The surgery required to place internal component 102 is much safer and easier than those used in some conventional systems that include power sources. For example, internal component 102 does not include a battery or other power source itself, which reduces the potential for corrosion or shocks at the implanted device. Rather, internal component 102 provides electrical connections to whichever muscles and/or nerves a physician or surgeon wishes to stimulate to prevent OSA on one end and configured to couple to a removable and replaceable power source at the other end in the user's mouth, at electrical coupler 104.

Electrical coupler 104 is coupled to internal component 102 both mechanically and electrically. In embodiments, electrical coupler 104 can be coupled to internal component 102 to provide power to stimulate the hypoglossal nerve and/or the genioglossus muscle, or others as described above. Electrical coupler 104 can be a simple insulated wire, in embodiments, or electrical coupler 104 can include a plurality of braided sub-wires or channels. In this way, in embodiments where internal component 102 is coupled to multiple nerves and/or muscles, power can be supplied to each through a separate sub-wire of electrical coupler 104. In some embodiments, electrical coupler 104 can also be used to deliver a coded digital or analog signal that indicates which of the muscles and/or nerves should be activated by internal component 102.

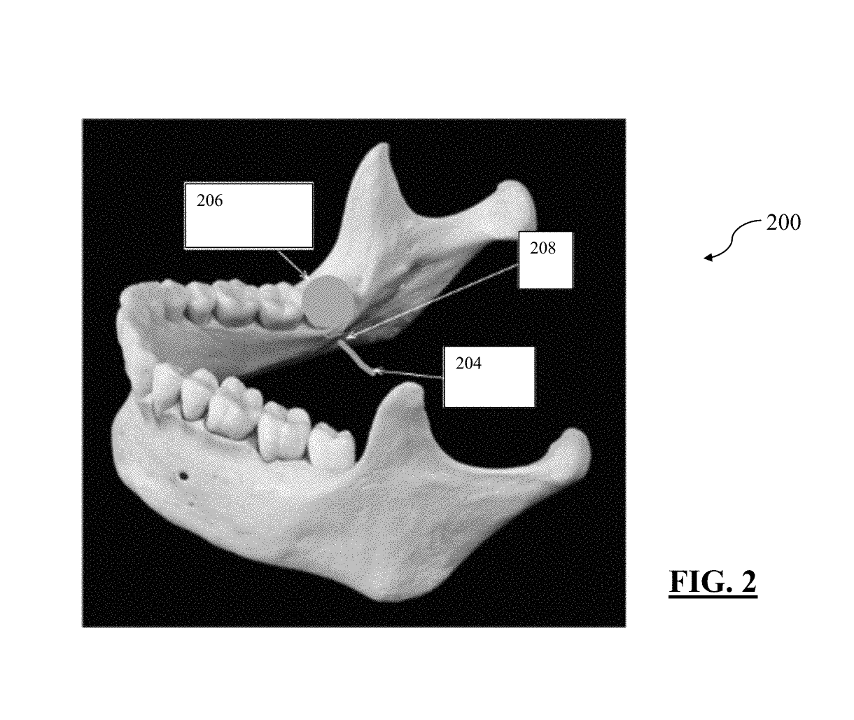

Electrical coupler 104 can be coupled to an external, removable, or otherwise non-implanted component, as described below in more detail. Such external components can be used to power internal component 102 via electrical coupler 104. Electrical coupler 104 extends from internal component 102 to some surface part of the patient, such as through a tooth as described in more detail below with respect to FIG. 2.

FIG. 2 is a perspective view of a surgically-altered jaw 200 of a patient suffering from OSA according to an embodiment. Surgically-altered jaw 200 includes access port 206, which is in electrical contact with electrical coupler 204.

Access port 206 can be any electrical or electronic coupling that facilitates contact between an external device and electrical coupler 204. In the embodiment shown in FIG. 2, access port 206 is a replacement tooth having an electrically conductive portion. In embodiments, access port 206 includes an electrically conductive portion and an electrically insulating portion. In various embodiments, the electrically insulating portion can be movable or deformable such that when access port 206 is in use, electrically conductive portions thereof are not exposed to the interior cavity of a mouth that includes jaw 200.

In this way, electricity and/or electrical signal can be transmitted from a remote source that is brought into contact with access port 206, to electrical coupler 204, to an internal component (e.g., internal component 102 of FIG. 1).

As shown in the embodiment in FIG. 2, access port 206 is positioned on the crown of a tooth, extending into surgically-altered jaw 200. The interface between access port 206 and electrical coupler 204 occurs within surgically altered jaw 200, at interface 208. Interface 208 is therefore kept dry and free from mechanical disturbances like bumping or pulling during regular activities of the patient.

Implanting the internal component (e.g., internal component 102 of FIG. 1) in a tooth is a fairly minor surgical procedure through surgically-altered jaw 200. In alternative embodiments access port 206 could be positioned elsewhere. For example, in some embodiments access port 206 could be a wire or sheet positioned in the mouth or jaw, or a series of such access ports. In embodiments, this access port or multiple access ports can be positioned at the base of the tongue or jaw such that there is easy access to a permanently or semi-permanently implanted, internal component.

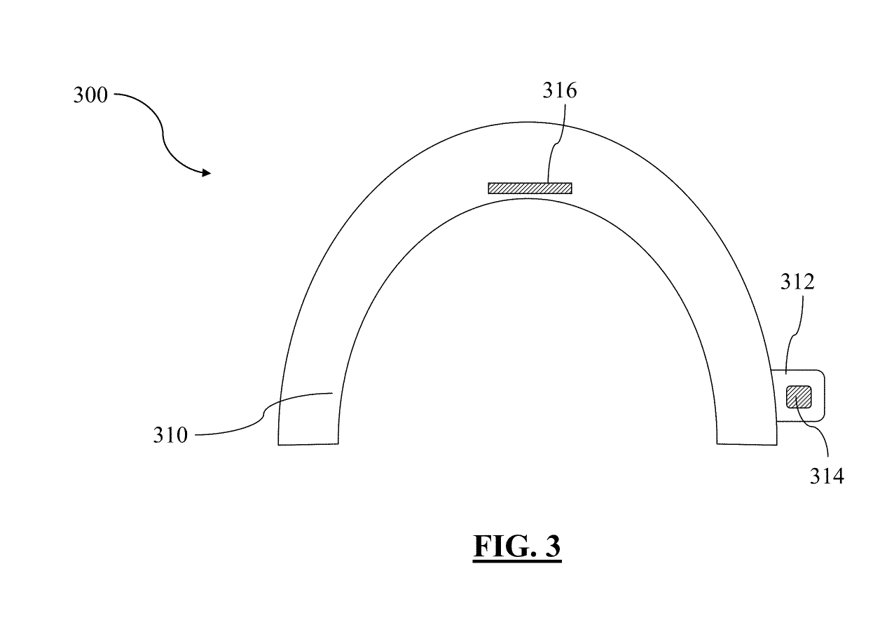

FIG. 3 is a bottom view of an external device 300. In the embodiment shown in FIG. 3, external device 300 includes retainer 310, bridge 312, access port counterpart 314, and charging port 316.

Retainer 310 can be sized and fitted to correspond to a jaw of a patient, such as surgically-altered jaw 200 of FIG. 2. Retainer 300 can include a power supply (not shown), such as a battery. This power supply can be incorporated into the body of retainer 310, in embodiments.

Bridge 312 and access port counterpart 314 are configured to engage with an access port, such as access port 206 of FIG. 2. Bridge 312 extends from retainer 310 to access port counterpart 314 and provides electrical connection from a power source such as a battery within retainer 310 to access port counterpart 314. In embodiments, access port 206 can be a socket and access port counterpart 314 can be a corresponding plug, for example. Those of skill in the art will recognize that various alternative structures could be used that provide electrical interconnection between the power source housed within retainer 310 and access port 206 when retainer 310 is positioned adjacent to jaw 200.

Charging port 316 is a port for charging of a battery or other power supply within retainer 310. In embodiments where retainer 310 includes a rechargeable battery, charging port 316 can be used to provide access for a cord or other charging device. In alternative embodiments, a non-rechargeable battery or power supply can be positioned within retainer 310. For such embodiments, it may be desirable to include an access panel or door to change out the battery or other power supply once it has been expended. In still further embodiments, retainer 310 can include a battery or other power supply that is configured for wireless charging, in which case no charging port 316 is necessary whatsoever.

In embodiments, retainer 310 further includes a processor or digital logic circuitry that can be used to transmit digital signals indicating a desired treatment pattern. In embodiments, the processor or digital logic circuitry can provide power at a predetermined level to any one or more of a series of sites at the genioglossus muscle or hypoglossal nerve. As previously described, in embodiments an internal component can be electrically coupled to one or more of a variety of muscles and/or nerves to stimulate different areas and prevent OSA. Therefore, in embodiments retainer 310 can send a signal indicative of whichever of these muscles and/or nerves should be stimulated at any given time. This can occur by providing a digital signal that can be decoded by a corresponding internal component, or in alternative embodiments access port counterpart 314 can include several conductive portions, each of which is in electrical communication with a different muscle/nerve or set of muscles/nerves.

In alternative embodiments, rather than a retainer 300, various other external or removable components could be used to provide power or signal to the implanted, internal components.

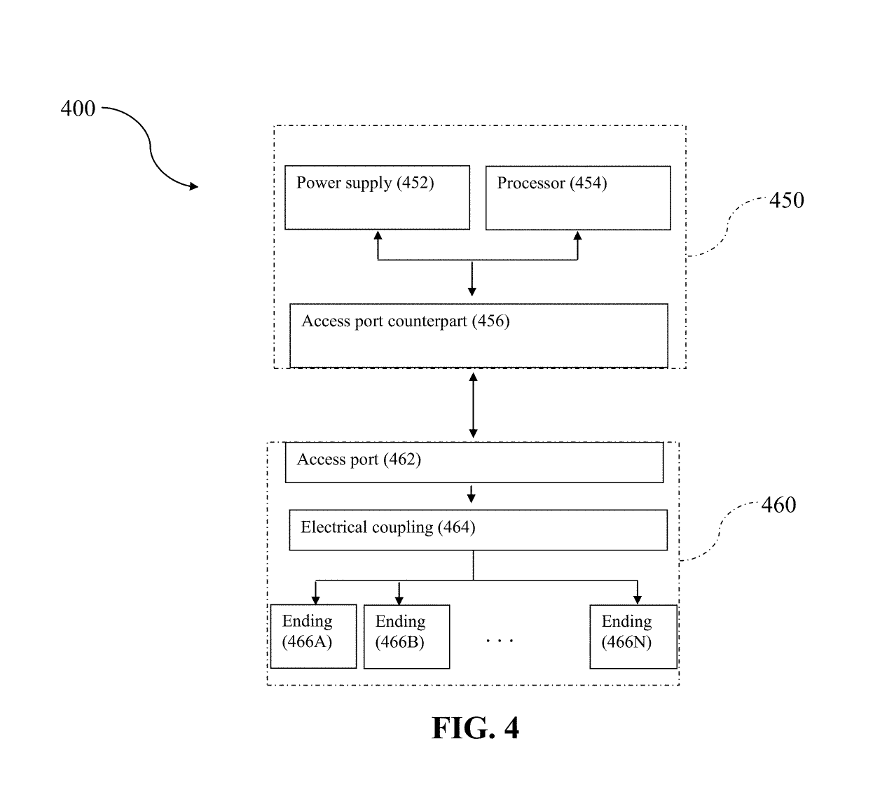

FIG. 4 shows a more general layout of a system 400 for providing relief from OSA. System 400 includes external component 450 and internal component 460. External component 450 includes power supply 452, processor 454, and access port counterpart 456. External component 450 can deliver power from power supply 452 to access port counterpart 456 based on commands from processor 454, as indicated by the arrows in FIG. 4.

Signal from access port counterpart 456 is delivered to access port 462 when external component 450 is placed into contact with internal component 460, as indicated in FIG. 4. Access port 462 receives electrical signal from access port counterpart 456 and transmits the electrical signal to electrical coupling 464. Electrical coupling 464, which can be surgically implanted to connect the access port 462 to one or more nerves or muscles, receives signal from access port 462 and delivers it to endings 466A, 466B, . . . 466N. In embodiments, each ending can correspond to a different muscle or nerve that a physician or surgeon identifies that should be stimulated to prevent obstruction of the airway due to OSA.

In embodiments, processor 454 can direct power from power supply 452 to an access port counterpart 456 that has multiple channels, such that endings 466A, 466B, . . . 466N receive electrical signal at different times from one another. In alternative embodiments, this same effect can be accomplished by sending a digital signal to access port counterpart 456, which can be decoded at access port 462 (possibly with the use of a separate processor or a multiplexor) to deliver power to endings 466A, 466B, . . . 466N.

Activating power delivery to different endings 466A, 466B, . . . 466N at different times can be beneficial because often one muscle cannot or should not be tensed by neurostimulation for an entire sleep cycle. Therefore, one or more muscles can be allowed to relax while others are stimulated, preventing complete obstruction of the airway at all times without requiring all the work to be done by one muscle (or few muscles).

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. §112(f) are not to be invoked unless the specific terms “means for” or “step for” are recited in a claim.

1. A system for preventing obstruction of an airway, the system comprising:
an internal component comprising:
an electrical coupling configured to extend to a muscle or nerve, and
an access port electrically connected to the electrical coupling; and
an external component comprising:
an access port counterpart configured to engage with the access port to provide an electrical connection, and
a power supply,
wherein the power supply is configured to provide power to the access port counterpart and the electrical connection, thereby powering the electrical coupling at the muscle or nerve.

2. The system of claim 1, wherein the external component is a retainer.

3. The system of claim 1, wherein the power supply is a battery.

4. The system of claim 1, wherein the external component further comprises a processor.

5. The system of claim 4, wherein the internal component comprises a plurality of electrical couplings each configured to extend to a corresponding muscle or nerve.

6. The system of claim 5, wherein the processor is configured to selectively power each of the plurality of electrical couplings.

7. The system of claim 1, wherein the access port is configured to be arranged in a surgically-altered jaw.

8. The system of claim 1, wherein the access port is configured to be arranged in a tooth.

9. The system of claim 5, wherein at least one of the electrical couplings extends to an ending at a hypoglossal nerve.

10. The system of claim 5, wherein at least one of the electrical couplings extends to an ending at a genioglossus muscle.

11. The system of claim 1, wherein the power supply comprises a rechargeable battery.

12. The system of claim 11, further comprising a charging port configured to facilitate charging of the battery.

13. The system of claim 1, wherein the rechargeable battery can be charged by wireless charging.

14. The system of claim 1, wherein the power supply comprises a disposable battery.

15. The system of claim 14, wherein the external component further comprises an access panel configured to permit replacement of the disposable battery.

16. The system of claim 1, wherein the external component comprises a bridge.

17. A kit for treating obstructive sleep apnea, the kit comprising:
an internal component comprising:
an electrical coupling configured to extend to a muscle or nerve, and
an access port electrically connected to the electrical coupling,
wherein the internal component is configured to be surgically implanted adjacent
at least one nerve or muscle in a patient; and
an external component comprising:
an access port counterpart configured to engage with the access port to provide an electrical connection, and
a power supply.

18. The kit of claim 17, wherein the electrical coupling comprises a plurality of endings configured to be surgically implanted adjacent to each of a corresponding one of the at least one nerve or muscle of the patient.

19. A retainer for use with a system for treating obstructive sleep apnea, the retainer comprising:
a body configured to engage with a user's teeth;
a bridge coupled to the body, the bridge comprising an electrical connector configured to engage with an access port;
a power supply configured to provide power to the electrical connector, the power supply comprising a battery; and
a processor configured to direct power from the power supply to the electrical connector at a predetermined level.

20. The retainer of claim 19, further comprising a charging port electrically coupled to the power supply such that the power supply can be recharged via the charging port.
